(19) United States
(12) Patent Application Publication
(10) Pub. No.: US 2023/0393090 A1
(43) Pub. Date: Dec. 7, 2023

(54) GAS SENSING APPARATUS

(71) Applicant: UNIVERSITEIT LEIDEN, Leiden (NL)

(72) Inventors: Gerard Jacob Pieter van WESTEN, Voorschoten (NL); Hans Michiel de BRUIJN, Leiden (NL); Gregory Fabrice SCHNEIDER, Den Haag (NL); Xuhan LIU, Leiden (NL); Erik Pieter van GEEST, Delft (NL); Sylvestre Antoíne BONNET, Amsterdam (NL)

(21) Appl. No.: 18/032,011

(22) PCT Filed: Oct. 13, 2021
(86) PCT No.: PCT/EP2021/078315

§ 371 Date: Apr. 14, 2023

(30) Foreign Application Priority Data
Oct. 15, 2020 (GB) 2016359.8

(51) Int. Cl. G01N 27/414; G01N 33/00

(52) U.S. Cl. CPC G01N 27/4141; G01N 27/04; G01N 33/0034

(57) ABSTRACT

There is provided a gas sensing apparatus (30) for sensing one or more analytes in a gas or gas mixture. The gas sensing apparatus (30) comprises: a plurality of sensors (32), each sensor (32) including a polymer layer (42), each polymer layer (42) made of a respective different type of chemically non-selective or semi-selective polymer; and a measurement device (38) configured to measure a change in parameter of each sensor (32) responsive to interaction of an analyte with the respective polymer layer (42).

This invention relates to a gas sensing apparatus, a method of using a gas sensing apparatus, a computer-implemented method of identifying one or more analytes in a gas or gas mixture and a computer program.

It is known to use a sensing device with a selective functionalised layer to sense a specific analyte in a sample.

According to a first aspect of the invention, there is provided a gas sensing apparatus for sensing one or more analytes in a gas or gas mixture, the gas sensing apparatus comprising:
a plurality of sensors, each sensor including a polymer layer, each polymer layer made of a respective different type of chemically non-selective or semi-selective polymer; and
a measurement device configured to measure a change in parameter of each sensor responsive to interaction of an analyte with the respective polymer layer.

The different types of chemically non-selective or semi-selective polymers have different chemical compositions. A chemically non-selective or semi-selective material has the ability to chemically interact with a wide range of analytes, as opposed to a chemically selective material that can only interact with a specific type of analyte.

The gas sensing apparatus of the invention comprises a set of sensors, typically arranged as an array of sensors, each of which is coated with a respective polymer layer. By virtue of each polymer layer being made of a respective different type of chemically non-selective or semi-selective polymer, each sensor responds in a unique and highly predictable way to chemical vapours, as determined by its respective polymer layer coating. The exact response of each sensor depends on the chemical nature of both the polymer layer coating and the vapour.

The provision of multiple sensors in the gas sensing apparatus of the invention enables combination of the measurements from the multiple sensors to provide information in the form of a respective unique chemical fingerprint that corresponds to the presence of the or each analyte in the gas or gas mixture. The chemical non-selectivity/semi-selectivity of the polymer layers enables the gas sensing apparatus to be used for sensing a wide range of analytes across a wide range of gases and gas mixtures, as opposed to the limited sensing range of chemically selective materials that can only sense a specific type of analyte.

The responsiveness of the polymer layers to chemical vapours together with the combination of the responses to form a unique chemical fingerprint for the or each analyte in the gas or gas mixture not only enables instantaneous and simultaneous sensing following exposure to the gas or gas mixture, thus resulting in more rapid and accurate real-time measurements to reduce overall sensing times but also permits a compact and portable configuration of the gas sensing apparatus by obviating a need for costly, complex and bulky equipment to physically separate the analytes in a gas mixture in order to sense the presence of each analyte.

In contrast, in order to enable separate detection of individual analytes in a gas mixture, gas chromatography requires the analytes to pass through a long separation column before they reach a detector, thus taking up space and increasing overall sensing times.

In embodiments of the invention, each sensor may include a sensing layer coated with the corresponding polymer layer, and the measurement device may be configured to receive an electrical signal from each sensor, the measurement device configured to measure a change in electrical parameter of each sensor responsive to interaction of an analyte with the respective polymer layer.

The electrical parameter may be, but is not limited to, electrical resistance, electrical capacitance or electrical impedance. The change in electrical parameter of each sensor responsive to the interaction of an analyte with the respective polymer layer may involve a change in electrical parameter of each sensing layer responsive to a change in dipole moment of the respective polymer layer due to the interaction of the respective analyte and the respective polymer layer. The change in electrical parameter of each sensor responsive to the interaction of an analyte with the respective polymer layer may involve a change in electrical capacitance or electrical impedance of the polymer layer. The change in electrical parameter of each sensor responsive to the interaction of an analyte with the respective polymer layer may involve electrical gating to, for example, look for a shift in the Dirac peak of the sensing layer.

This enables the gas sensing apparatus to be used with conventional means and equipment for measuring electrical parameters, thus removing the need for customised measurement equipment. As a result, this flexibility enables the gas sensing apparatus to be used in a wide range of applications and environments.

Furthermore, using the polymer layers as coatings for the respective sensing layers increases the robustness of the sensors and reduces electronic noise, thus significantly improving the reliability and performance of the gas sensing apparatus.

The sensing layer may be a graphene-based sensing layer. In a preferred embodiment of the invention, each sensor includes a graphene field-effect transistor, and the graphene-based sensing layer is built into the graphene field-effect transistor.

The thinness of graphene layers provides a high surface to volume ratio that is conducive to a highly sensitive sensing capability of the sensors. This is because the sensing process in a given graphene-based sensor of the invention occurs through interactions at the surface of the sensing layer, which in turn means that the sensing performance of the graphene-based sensor increases with surface to volume ratio. This in combination with the high travel speeds of electrons in graphene and the high sensitivity of the graphene-based sensors to their environment through exposed p-orbitals that are perpendicular to the graphene plane renders the graphene-based sensors highly suitable to increase the sensing performance of the gas sensing apparatus.

It will be appreciated that any type of graphene-based material, or a combination of different types of graphene-based materials, may be used to fabricate the graphene-based sensing layer.

Preferably the graphene-based sensing layer is a graphene monolayer. The graphene monolayer may be obtained by synthesis on a suitable substrate, such as copper, via chemical vapour deposition (CVD) under suitable temperature conditions. Preferably the graphene is pure in the sense that it has no defects that is measurable by Raman spectroscopy.

In further embodiments of the invention, the measurement device may include a detector configured to measure a change in mechanical parameter and/or a change in structural parameter of each polymer layer responsive to interaction of an analyte with the respective polymer layer. In such embodiments, the detector may include a spectrometer, such as an infra-red spectrometer or a Raman spectrometer.

Measuring mechanical and/or structural changes in the polymer layers provide a reliable means of detecting the interaction of an analyte with the respective polymer layer because each polymer layer interacting with the analyte will undergo mechanical and/or structural changes that are different from each other polymer layer.

It will be appreciated that any type of polymer can be used to fabricate the polymer layer of each sensor. The selection of each polymer depends on the sensing and cost requirements of the gas sensing apparatus.

Each polymer may be selected from, but is not limited to, a group consisting of: polymethyl methacrylate (PMMA); cellulose acetate butyrate (CAB); tetrafluoroethylene-perfluoro-3,6-dioxa-4-methyl-7-octenesulfonic acid copolymer, also known as Nafion™ 117.

Preferably each polymer can be applied as a thin film (e.g. a film having a thickness under 500 nm) to form the polymer layer.

Each polymer may be spin-coatable to form the polymer layer.

Each polymer may be a transfer polymer.

In embodiments in which each sensor includes a sensing layer and in which the sensing layer is fabricated on a separate substrate or surface, the transfer polymer may be used to transfer the sensing layer to a substrate or surface of the gas sensing apparatus and may be retained to function as the chemically non-selective or semi-selective polymer for the polymer layer. Using a transfer polymer as the chemically non-selective or semi-selective polymer for each polymer layer helps the polymer and sensing layers to stay intact during fabrication, thus improving the sensor's capability to provide homogenous and highly predictable responses to chemical vapours, but also removes the need for an additional polymer or sensing material deposition step, thus reducing fabrication time and increasing reproducibility of the gas sensing apparatus.

The gas sensing apparatus may include a housing, e.g. a tubular housing, arranged to enclose the plurality of sensors, the housing including: a gas inlet for permitting the gas or gas mixture to enter the housing; and a gas outlet for permitting the gas or gas mixture to exit the housing. In embodiments of the invention that are configured to measure a change in electrical parameter of each sensor responsive to interaction of an analyte with the respective polymer layer, the sensors may be electrically accessed through electrical connections extending through a wall of the housing from the sensors to an exterior of the housing or through wireless connections such as Bluetooth or Near-field Communication.

The provision of the housing to enclose the plurality of sensors provides a reliable means of configuring the gas sensing apparatus as a compact and portable arrangement. The gas inlet and outlet readily enable the flow of the gas or gas mixture into and out of the housing in order to expose the sensors to the gas or gas mixture. This may be carried out by, for example, the gas sensing apparatus including a gas delivery device configured to selectively inject the gas or gas mixture into the housing via the gas inlet.

The gas delivery device may be configured to automatically inject a sample in the form of a gas or gas mixture, or different gases or gas mixtures, at specified intervals into the housing. The compatibility of the gas sensing apparatus with an automated gas injection device provides a reliable system of gathering large data sets and wide screening of chemical vapours that enable identification of the chemical fingerprints of these vapours from the combined information of the responses of the individual sensors in the gas sensing apparatus.

In embodiments of the invention, the measurement device may include a processor and memory including computer program code, the memory and computer program code configured to, with the processor, enable the measurement device at least to combine the measured changes in parameter of the sensors so as to generate a chemical fingerprint of the or each analyte in the gas or gas mixture.

In further embodiments of the invention, the measurement device may include a processor and memory including computer program code, the memory and computer program code configured to, with the processor, enable the measurement device at least to analyse the measured changes in parameter of the sensors to identify the or each analyte in the gas or gas mixture.

Configuration of the measurement device in this manner provides automatic generation of the chemical fingerprint of the or each analyte in the gas or gas mixture and automated analysis of the measured changes in parameter of the sensors to identify the or each analyte in the gas or gas mixture, which is particularly beneficial for gathering large data sets and wide screening of chemical vapours.

In such embodiments, the memory and computer program code may be configured to, with the processor, enable the measurement device at least to analyse the measured changes in parameter of the sensors to identify the or each analyte in the gas or gas mixture by providing the measured changes in parameter of the sensors as input to a machine learning model and identifying the or each analyte in the gas or gas mixture based on an output of the machine learning model.

Configuration of the measurement device in this manner enables the gas sensing apparatus to have machine-learned selectivity that in turns facilitates characterisation and quantification of chemical vapours in unknown samples, such as disease-specific species in breath analysis samples. Specifically, training sets based on the unique chemical fingerprints corresponding to the presence of the or each analyte in the gas or gas mixture can be used to train the gas sensing apparatus through supervised learning.

According to a second aspect of the invention, there is provided a method of using a gas sensing apparatus to sense one or more analytes in a gas or gas mixture, the method comprising the steps of:
providing a plurality of sensors, each sensor including a polymer layer, each polymer layer made of a respective different type of chemically non-selective or semi-selective polymer;
exposing the plurality of sensors to the gas or gas mixture;
measuring a change in parameter of each sensor responsive to interaction of an analyte with the respective polymer layer.

The features and advantages of the first aspect of the invention and its embodiments apply mutatis mutandis to the features and advantages of the method of the second aspect of the invention and its embodiments.

When each sensor includes a sensing layer coated with the corresponding polymer layer, the method may include the step of measuring a change in electrical parameter of each polymer layer responsive to interaction of an analyte with the respective polymer layer.

In the method of the invention, the sensing layer may be a graphene-based sensing layer. In a preferred embodiment of the method of the invention, each sensor includes a graphene field-effect transistor, and the graphene-based sensing layer is built into the graphene field-effect transistor.

The method may include the step of measuring a change in mechanical parameter and/or a change in structural parameter of each polymer layer responsive to interaction of an analyte with the respective polymer layer.

In the method of the invention, each polymer may be selected from, but is not limited to, a group consisting of: PMMA; CAB; Nafion™ 117.

In the method of the invention in which each sensor includes a sensing layer, each polymer may be a transfer polymer, and wherein the step of providing the plurality of sensors may include using each transfer polymer to transfer the respective sensing layer from a first substrate or surface to a second substrate or surface, wherein the second substrate or surface forms part of the gas sensing apparatus.

In the method of the invention, the step of providing the plurality of sensors may include taking each sensor from a respective different batch of sensors, wherein the polymer layer of each sensor in the respective different batch of sensors is made of a same type of chemically non-selective or semi-selective polymer layer.

The method of the invention may include the step of enclosing the plurality of sensors in a housing, e.g. a tubular housing, the housing including: a gas inlet for permitting the gas or gas mixture to enter the housing; and a gas outlet for permitting the gas or gas mixture to exit the housing.

The method may include the step of injecting the gas or gas mixture into the housing via the gas inlet.

The method of the invention may include the step of combining the measured changes in parameter of the sensors so as to generate a chemical fingerprint of the or each analyte in the gas or gas mixture.

The method of the invention may include the step of analysing the measured changes in parameter of the sensors to identify the or each analyte in the gas or gas mixture.

The method of the invention may include the step of analysing the measured changes in parameter of the sensors to identify the or each analyte in the gas or gas mixture by providing the measured changes in parameter of the sensors as input to a machine learning model and identifying the or each analyte in the gas or gas mixture based on an output of the machine learning model.

According to a third aspect of the invention, there is provided a computer-implemented method of identifying one or more analytes in a gas or gas mixture, the method comprising:
collecting a set of data by carrying out the method according to any one of the second aspect of the invention and its embodiments, wherein the collected set of data includes the measured changes in parameter of the sensors;
creating a training set including the collected set of data;
training a machine learning model using the training set;
identifying the or each analyte in the gas or gas mixture based on an output of the machine learning model.

The features and advantages of the first and second aspects of the invention and their embodiments apply mutatis mutandis to the features and advantages of the computer-implemented method of the third aspect of the invention and its embodiments.

According to a fourth aspect of the invention, there is provided a computer program comprising computer code configured to perform the method of any one of the third aspect of the invention and its embodiments.

The features and advantages of the first, second and third aspects of the invention and their embodiments apply mutatis mutandis to the features and advantages of the computer program of the fourth aspect of the invention and its embodiments.

In embodiments of the invention the measurement device may be, may include, may communicate with or may form part of one or more of an electronic device, a portable electronic device, a portable telecommunications device, a mobile phone, a personal digital assistant, a tablet, a phablet, a desktop computer, a laptop computer, a server, a cloud computing network, a smartphone, a smartwatch, smart eyewear, and a module for one or more of the same.

A non-limiting application of the invention is the use of the gas sensing apparatus as a point-of-care device in clinics and hospitals for the screening of patients by breath analysis, which can be used to aid health diagnosis. It may be possible to individually identify some or all species in the breath from the combined information obtained from the sensors. The ability of the gas sensing apparatus to provide the required gas sensing information provides the option of rapid analysis, thus turning the invention into a tool for fast screening of patients. Due to the relatively low cost and rapid sensing of the gas sensing apparatus, tests can be carried out on the spot by a general practitioner to get an indication of the well-being of the patient and obtain a better understanding for follow-up tests or treatment. This obviates the need for more costly equipment, such as gas chromatography equipment, that would be cost-prohibitive for a general practitioner to operate on site.

Other non-limiting applications for the gas sensing apparatus of the invention include, but are not limited to:
regular or continuous measurement of air quality in buildings and other enclosed environments;
ethene concentration sensing for detecting and monitoring ripening processes of fruit during their transportation and storage.

It will be appreciated that the use of the terms “first” and “second”, and the like, in this patent specification is merely intended to help distinguish between similar features (e.g. the first and second substrates or surfaces, etc.), and is not intended to indicate the relative importance of one feature over another feature, unless otherwise specified.

Within the scope of this application it is expressly intended that the various aspects, embodiments, examples and alternatives set out in the preceding paragraphs, and the claims and/or the following description and drawings, and in particular the individual features thereof, may be taken independently or in any combination. That is, all embodiments and all features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

Preferred embodiments of the invention will now be described, by way of non-limiting examples, with reference to the accompanying drawings in which:

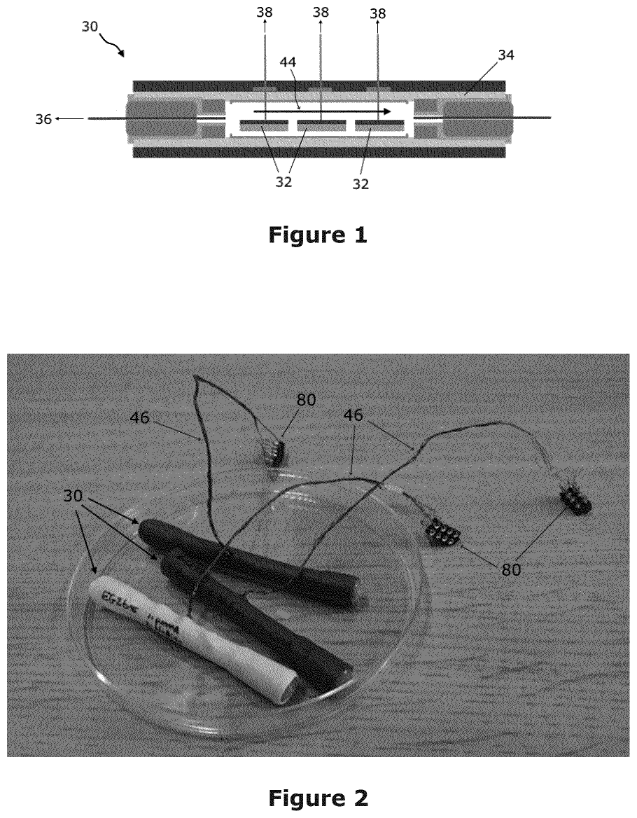

FIGS. 1 and 2 show a gas sensing apparatus according to an embodiment of the invention;

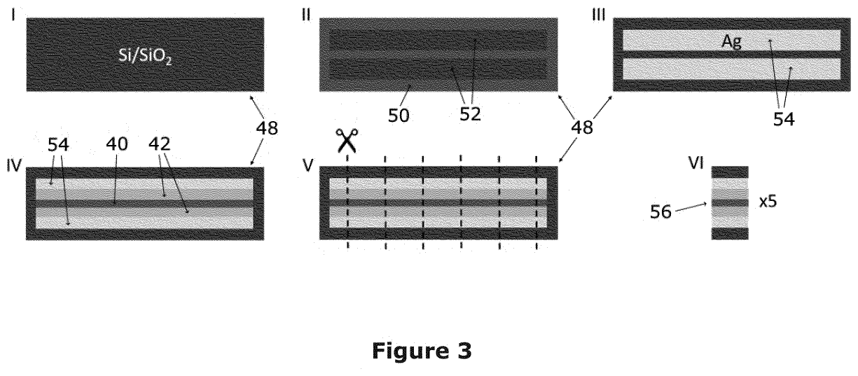

FIGS. 3 to 5 show a fabrication process of the gas sensing apparatus of FIGS. 1 and 2;

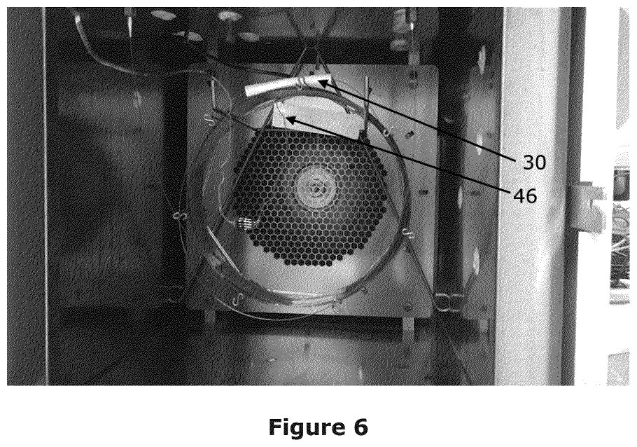

FIG. 6 shows a testing setup of the gas sensing apparatus of FIGS. 1 and 2;

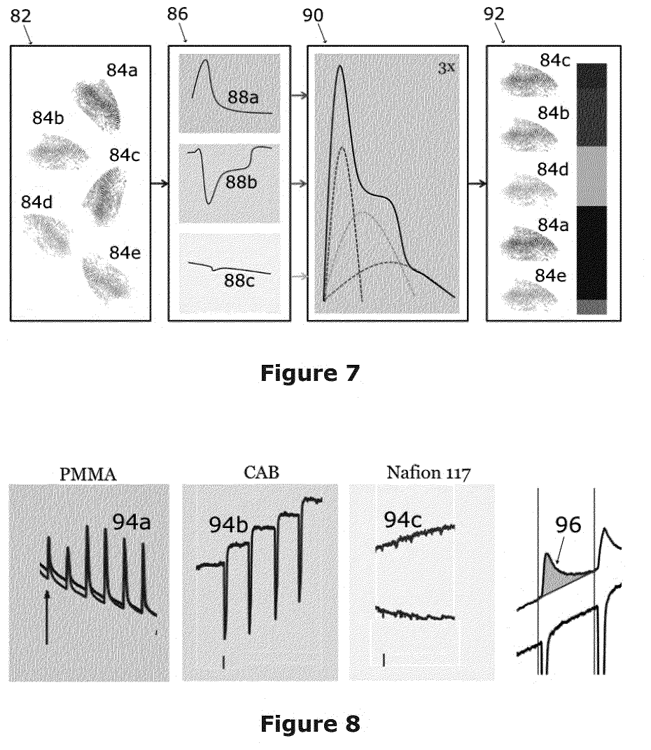

FIG. 7 shows an operation of the gas sensing apparatus of FIGS. 1 and 2;

FIGS. 8 to 10 show measurements of the presence of one or more analytes in a gas or gas mixture by the gas sensing apparatus of FIGS. 1 and 2;

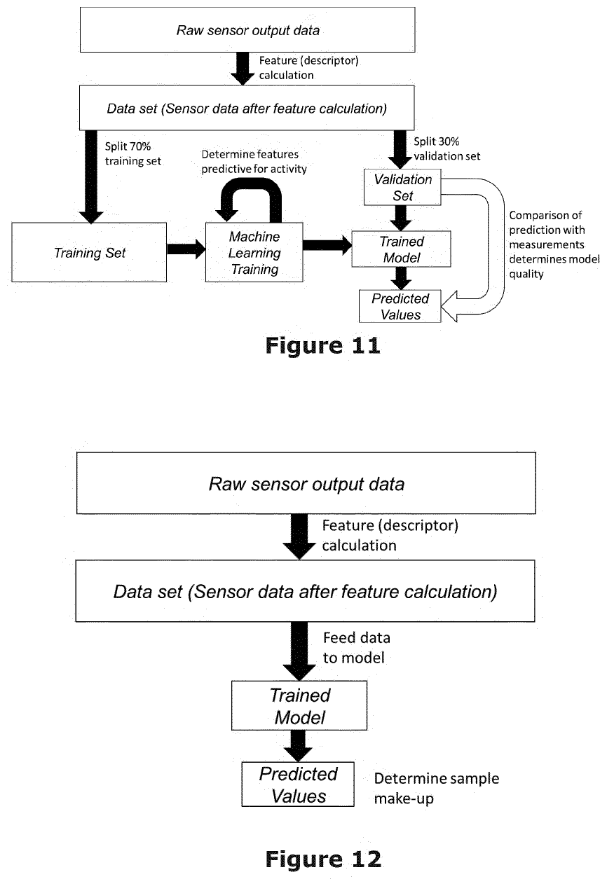

FIGS. 11 to 13 illustrate an exemplary machine learning process for use with the gas sensing apparatus of FIGS. 1 and 2;

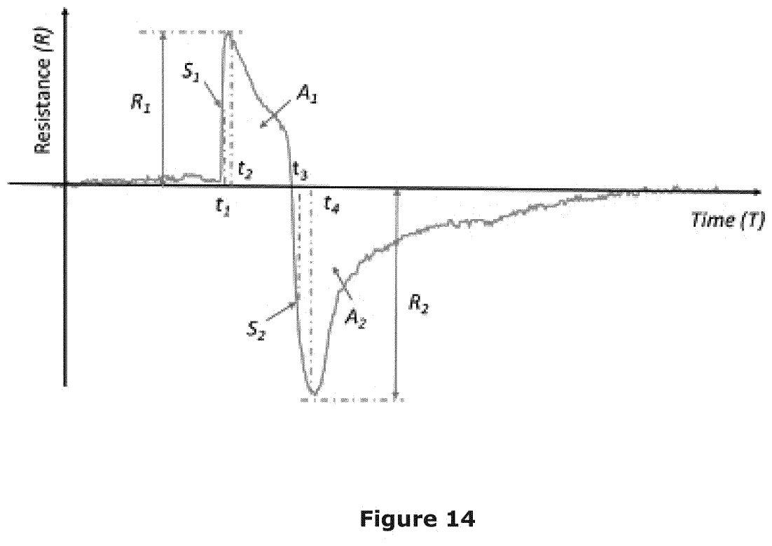

FIG. 14 shows measured electrical resistance data obtained by the gas sensing apparatus of FIGS. 1 and 2; and

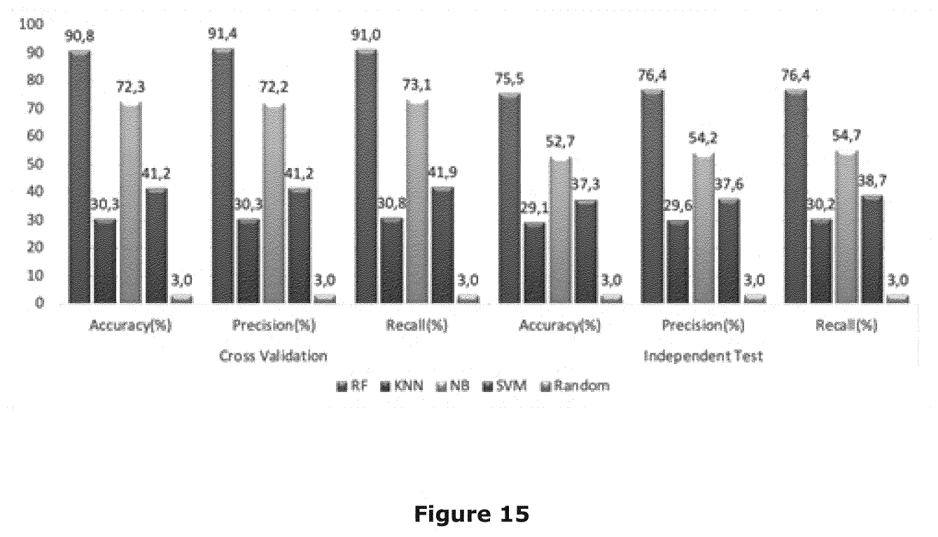

FIG. 15 shows the performance of the machine learning model of FIGS. 11 to 13.

The figures are not necessarily to scale, and certain features and certain views of the figures may be shown exaggerated in scale or in schematic form in the interests of clarity and conciseness.

A gas sensing apparatus according to an embodiment of the invention is shown in FIGS. 1 and 2 and is designated generally by the reference numeral 30.

The gas sensing apparatus 30 is configured for sensing one or more analytes in a gas or gas mixture. The gas sensing apparatus 30 comprises a plurality of sensors 32, a housing 34, a gas delivery device 36 and a measurement device 38. In the embodiment shown, the gas sensing apparatus 30 has three sensors 32. It will however be appreciated that the gas sensing apparatus 30 may have any number of a plurality of sensors 32.

Each sensor 32 includes a sensing layer 40 coated with a polymer layer 42, where the sensing layer 40 is a graphene-based sensing layer 40 that is built into a graphene field-effect transistor. Each polymer layer 42 is made of a respective different type of chemically non-selective or semi-selective polymer. Non-limiting examples of non-selective and semi-selective polymers are described later and elsewhere in this specification.

The housing 34 is in the form of a tube that encloses the plurality of sensors 32. At one end of the tube, the housing 34 includes a gas inlet for permitting the gas or gas mixture to enter the housing 34. At the other end of the tube, the housing 34 includes a gas outlet for permitting the gas or gas mixture to exit the housing 34. The arrow 44 in FIG. 1 indicates the flow direction of the gas or gas mixture through the housing 34. Electrical wires 46 extend through a wall of the housing 34 and are connected to the sensors 32 in order to permit electrical access to the sensors 32 from an exterior of the housing 34.

Fabrication of the gas sensing apparatus 30 is described as follows with reference to FIGS. 3 to 5.

FIG. 3 shows a step-by-step process of manufacturing daughter chips that can be processed into sensors 32. Firstly, a long silicon mother chip 48 (approximately 40 mm by 10 mm) is cleaned and treated with O₂ plasma (I). A mask material 50 (such as tape or photoresist) is then placed onto the surface of the mother chip 48, leaving exposed areas 52 for electrodes (II). A silvering solution is applied to the mother chip so as to deposit metallic silver at the exposed areas 52, which results in formation of silver electrodes 54 after removal of the mask and cleaning (III). Next, a graphene monolayer 40 is formed on another substrate (e.g. using conventional graphene formation methods) before being transferred to the mother chip 48 with the assistance of a transfer polymer 42 (IV). The graphene layer 40 is arranged between and in contact with the silver electrodes 54. The transfer polymer 42 is retained on the mother chip 48 as a coating over the graphene layer 40. After drying and heating, the mother chip 48 is cut by introducing defects at its edge and applying pressure (V) to produce several daughter chips 56 (approximately 3 mm by 10 mm) that are to be processed into sensors 32 (VI).

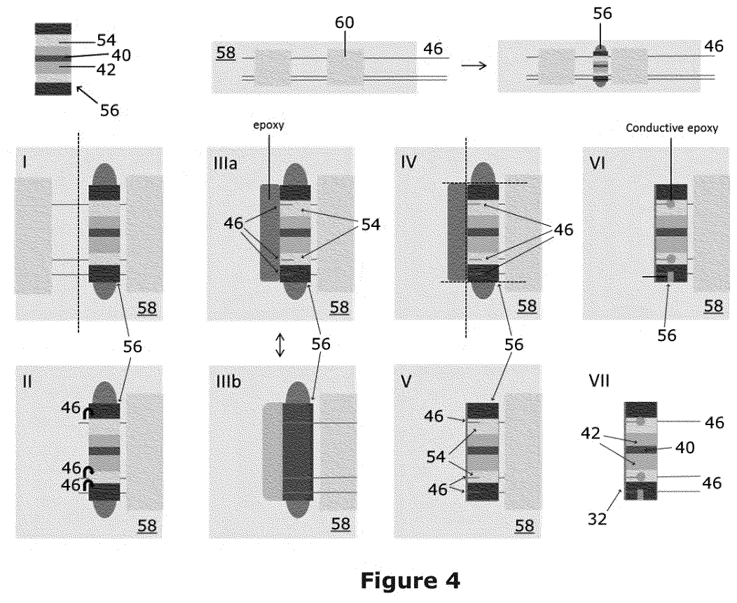

FIG. 4 shows a step-by-step process of manufacturing individual sensors 32 from the daughter chips 56. First, a daughter chip 56 is mounted on a glass slide 58 that has 3 copper wires 46, with tape 60 securing the copper wires 46 to the glass slide 58. The daughter chip 56 is placed on the wires 46 and glued to the glass slide 58 with epoxy resin. The wires 46 are cut 1 mm away from the edge of the daughter chip 56 using a razor blade (I; seen from top) and bent upwards, with 2 wire ends over the electrodes 54 of the daughter chip 56 and one close to the edge of the daughter chip 56 (II). The bent wires 46 are fixed in position with epoxy resin (IIIc), and the resin flows under the daughter chip 56 as well (IIIb, seen from below). Excess epoxy is then removed with a razor blade (IV), where the razor blade is placed as close as possible to the edges of the daughter chip 56 without damaging the wires 46 (V). Next, the wires 46 are connected to the electrodes 54 (2×) and to the back side of the daughter chip 56 (1×) using a silver-based conductive epoxy (VI). Finally, the daughter chip 56 is removed carefully from the glass slide 58, giving the finished individual sensor 32 (VII).

The processes in FIGS. 3 and 4 are repeated for different batches of daughter chips 56 using different transfer polymers 42. A daughter chip 56 is taken from each different batch of daughter chips 56 to produce a set of sensors 32 in which each individual sensor 32 has a polymer layer 42 made of a respective different chemically non-selective or semi-selective polymer. These sensors 32 are then used in the assembly of the gas sensing apparatus 30.

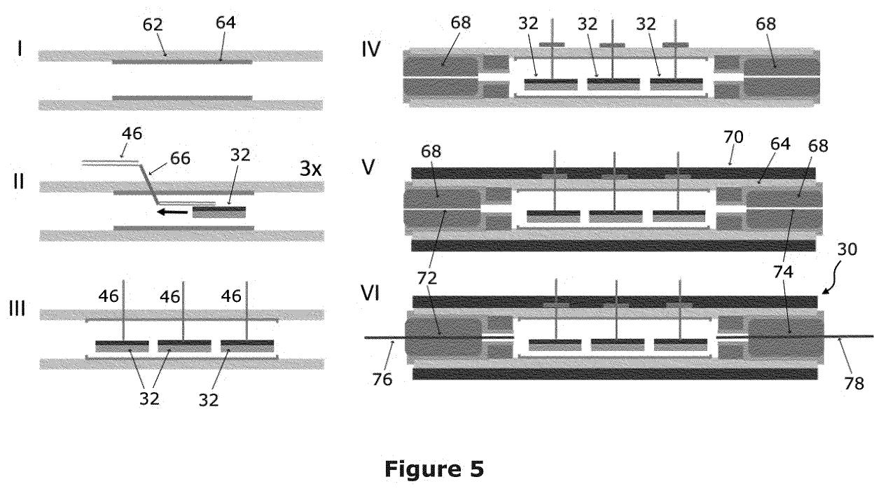

FIG. 5 shows a step-by-step process of assembling the gas sensing apparatus 30. A polytetrafluoroethylene (PTFE) tube 62 is inserted in a bore of a silicone tube 64, with the ends of the PTFE tube 62 spaced about 2 cm from the respective ends of the silicone tube 64. The individual sensors 32 are inserted into the PTFE tube 62, and the wires 46 are run through wire holes in the walls of the tubes 62,64 with a needle 66 (II). The individual sensors 32 are lined up in the PTFE tube 62 (III). The ends of the silicone tube 62 are cut to fit sensor caps 68. The sensor caps 68 are installed at the ends of the silicone tube 64 and are fixed and sealed in place with epoxy resin. The wire holes are patched with epoxy resin (IV). A thermoplastic sleeve 70 is placed around the silicone tube and fixed in place by shrinkage using a heat gun (V). The thermoplastic sleeve 70 also includes holes for receiving the wires 46. Inlet and outlet holes 72,74 in the sensor caps 68 define the gas inlet and outlet. In use, inlet and outlet gas conduits 76,78 are respectively inserted into the inlet and outlet holes 72,74.

The inlet gas conduit 76 is connected to the gas delivery device 36 in order to permit the gas delivery device 36 to inject a gas or gas mixture into the housing 34 and thereby expose the sensors 32 to the gas or gas mixture. FIG. 6 shows the gas sensing apparatus 30 connected to an automated gas injection system, which comprises the gas delivery device, so that in use the gas delivery device 36 is operable to inject a gas or gas mixture into the housing 34 via the gas inlet.

The measurement device 38 includes a processor and memory including computer program code. The memory and computer program code are configured to, with the processor, enable the measurement device 38 to carry out various processing functions. The measurement device 38 may be, may include or may form part of one or more of an electronic device, a portable electronic device, a portable telecommunications device, a mobile phone, a personal digital assistant, a tablet, a phablet, a desktop computer, a laptop computer, a server, a cloud computing network, a smartphone, a smartwatch, smart eyewear, and a module for one or more of the same. It will be appreciated that references to a memory or a processor may encompass a plurality of memories or processors.

FIGS. 1 and 6 show the electrical wires 46 that are connected to the measurement device 38 to enable it to receive an electrical signal from each sensor 32. The electrical wires 46 may be combined into a single connector 80 to configure the gas sensing apparatus 30 as a plug-and-play apparatus.

FIG. 7 shows a typical workflow of the gas sensing apparatus 30 to generate a chemical fingerprint that can be used to identify and quantify analytes in a gas mixture.

In a first step 82, the sensors 32 are exposed to the gas mixture that comprises a plurality of different vapours 84. In a second step 86, the resultant change in electrical resistance 88a,88b,88c of each sensor 32 responsive to interaction of the analytes with the respective polymer layer 42 is sensed through the sending of the resultant electrical signals from the sensors 32 to the measurement device 38. In a third step 90, the measurement device 38 filters the received signals, e.g. through deconvolution. Finally, in a fourth step 92, the measurement device 38 combines the filtered electrical resistance measurements to generate chemical fingerprint information about the gas mixture. The chemical fingerprint information is then compared with a chemical fingerprint database to identify and quantify the analytes in the gas mixture. The filtering of the received signals prior to their combination is optional.

FIG. 8 illustrates the response of the sensors 32 to acetone, where the polymer layers 42 of the sensors 32 are respectively coated with three different chemically non-selective polymers, PMMA, CAB, Nafion™ 117. It can be seen that the provision of the polymer layer 42 has a large effect on the responses 94a,94b,94c by the sensors 32 upon exposure to acetone. The far right image of FIG. 8 shows an integration peak 96 in the electrical resistance profile as a result of the acetone vapour passing over a given sensor 32. The combined information of the peak areas from the different sensors 32 can be used as a chemical fingerprint for acetone.

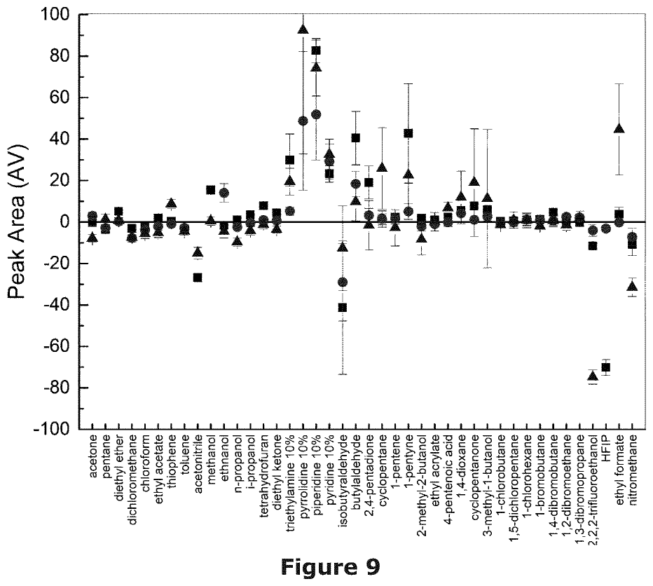

FIG. 9 shows the chemical fingerprints of different gaseous compounds based on average peak area, obtained from normalised electrical resistance data (R/R0×100). HFIP in FIG. 9 refers to 1,1,1,3,3,3-hexafluoro-2-propanol. The squares represent the data obtained from the PMMA-coated sensor 32, the circles represent the data obtained from the Nafion™ 117-coated sensor 32 and the triangles represent the data obtained from the CAB-coated sensor 32. The peak areas shown in FIG. 8 are averages of 4 data points per species, except for acetone for which the peak area is an average of 6 data points. It can be seen that unique chemical fingerprints can be obtained for a wide range of gaseous compounds using the gas sensing apparatus 30 of the invention.

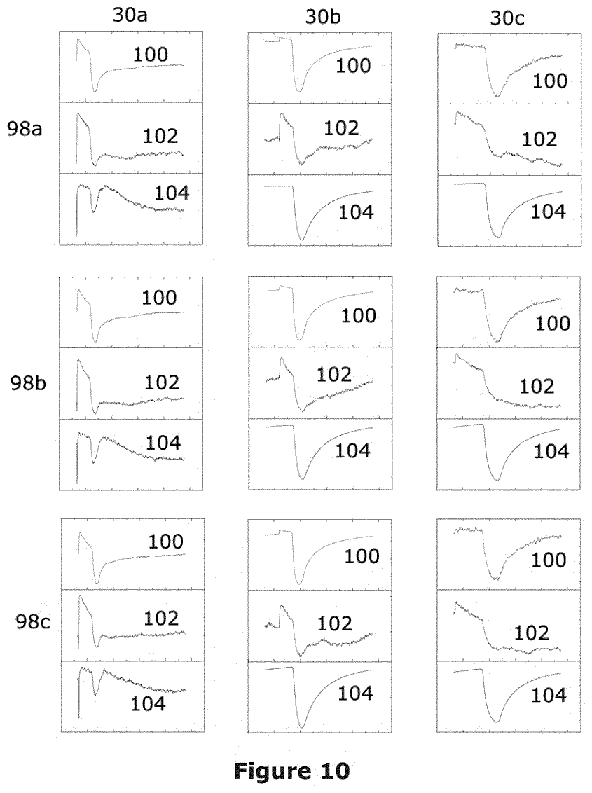

Another way of using the electrical resistance information from the sensors 32 to generate a chemical fingerprint for a gas mixture is shown in FIG. 10. FIG. 10 shows the variation of electrical resistance with time for three gas sensing apparatus when sequential injections 98a,98b,98c of HFIP are applied to each gas sensing apparatus 30a,30b,30c. Each gas sensing apparatus 30a,30b,30c has a

PMMA-coated sensor 32, a Nafion™ 117-coated sensor 32 and a CAB-coated sensor 32. The top row shows the respective responses 102,104,106 for the PMMA-coated sensor 32, the Nafion™ 117-coated sensor 32 and the CAB-coated sensor 32 across the different gas sensing apparatus 30a,30b,30c for a first injection 98a of the HFIP. The middle row shows the respective responses 102,104,106 for the PMMA-coated sensor 32, the Nafion™ 117-coated sensor 32 and the CAB-coated sensor 32 across the different gas sensing apparatus 30a,30b,30c for a second injection 98b of the HFIP. The bottom row shows the respective responses 102,104,106 for the PMMA-coated sensor 32, the Nafion™ 117-coated sensor 32 and the CAB-coated sensor 32 across the different gas sensing apparatus 30a,30b,30c for a third injection 98c of the HFIP. Each graph in FIG. 10 has electrical resistance as its y-axis and time as its x-axis.

The changes in electrical resistance of the sensors 32 were measured simultaneously due to the arrangement of the sensors 32 in each gas sensing apparatus 30a,30b,30c.

For each gas sensing apparatus 30a,30b,30c, the sequential injections 98a,98b,98c of the same HFIP compound resulted in very similar electrical resistance vs time profiles of the responses 102,104,106 of the sensors 32, thus showing the reproducibility of the sensing performance of the gas sensing apparatus 30a,30b,30c. Generally the electrical resistance vs time profiles of the responses 102,104,106 of the sensors 32 were similar, with some variation, across the different gas sensing apparatus 30a,30b,30c, thus showing the consistency of the gas sensing apparatus design and the fabrication protocol.

In view of the foregoing the inventors observed that the sensors 32 of the gas sensing apparatus 30 of the invention show reproducible and predictable responses to chemical vapours. The automatic injection of the gas or gas mixture into the gas sensing apparatus 30 removed the influence of human errors on the responses of the sensors 32.

Having an array of sensors 32 with graphene-based sensing layers 40 coated with different transfer polymers 42 produces a gas sensing apparatus 30 that can produce reliable chemical fingerprints of a wide range of chemical vapours. By using the transfer polymer 42 as the chemically non-selective or semi-selective polymer for each polymer layer 42, the polymer can be kept intact on the graphene layer 40 to improve the sensor's capability to provide homogenous and highly predictable responses to chemical vapours, especially due to the ability to control the quality and quantity of the transfer polymer 42. This is not the case for sensors that rely on the presence of polymer residues in unknown quantities and qualities, which would result in inhomogeneous and unpredictable responses to chemical vapours.

The type of transfer polymer 42 may be changed to modify the sensing performance of the sensor 32 and therefore the gas sensing apparatus 30. This is because the exact response of the sensor 32 is largely governed by the chemical nature of the polymer layer 42.

Furthermore the polymer layer 42 acting as a protective coating for the respective graphene layer 40 in each sensor 32 increases robustness of the sensors 32 and reduces electronic noise, which is not available for uncoated, exposed graphene sensors.

In other embodiments of the invention, the polymer layer may be made of a chemically non-selective or semi-selective polymer that is not the transfer polymer. For example, a chemically non-selective or semi-selective polymer may be deposited onto the graphene layer 40 after the transfer polymer is removed, or a chemically non-selective or semi-selective polymer may be deposited onto the graphene layer 40 that was formed on a substrate or surface of the gas sensing apparatus, instead of a separate substrate or surface.

The parallel construction of identical graphene sensing layers 40 coated with different polymers in the gas sensing apparatus 30 also offers a very versatile range of selectivity and price. In this regard the gas sensing apparatus 30 can be designed to have any number of a plurality of sensors 32 to meet specific sensing performance and cost requirements. For example, the number of sensors 32 may be increased to enhance the ability of the gas sensing apparatus 30 to distinguish between different gases or gas mixtures, while the number of sensors 32 may be decreased to reduce the overall cost of the gas sensing apparatus 30. This is because the use of the same fabrication protocol to make the individual sensors 32 of the gas sensing apparatus 30, with the exception of the choice of the respective chemically non-selective or semi-selective polymer, allows straightforward scale-up of the sensors 32 and therefore the gas sensing apparatus 30. For example, referring to FIGS. 3 and 4, multiple sensors 32 using the same polymer type are obtained by simply breaking them from the mother chip, giving minimal batch-to-batch variations and fast reproduction.

Critically the use of the multiple sensors 32 with different polymer layers 42 means that the selectivity of the gas sensing apparatus 30 is not limited to a single species, unlike conventional sensors that are coated with species-selective entities (e.g. enzymes). Such conventional sensors rely on proteins for detection of their biological binding partner, making them especially sensitive to this binding partner. However, such conventional sensors are not useful in determining the components of a mixture. Moreover, as with all biological materials, proteins are quite prone to damage, e.g. by UV irradiation, which greatly reduces the lifetime of the conventional sensors.

The selectivity of the gas sensing apparatus 30 together with the automated gas injection of the gas delivery device 36 can be used to construct a large database of individual responses of the sensors 32 to a large range of individual analytes for use in the determination and quantification of the composition of known and unknown gas mixtures. Obtaining selectivity through a combination of sensing information allows for cheaper sensing with a high diversity of uses because the gas sensing apparatus can be trained to identity species of interest and be optimised for specific samples.

The database can be used to create one or more training sets to train a machine learning model through supervised learning to enable the gas sensing apparatus 30 to identify and quantify one or more individual analytes through machine-learned pattern recognition. This is carried out by the measurement device 38 providing the training set(s) as input to the machine learning model and using an output of the machine learning model as the basis for identifying and quantifying the analyte(s) in the gas or gas mixture. New chemical fingerprint data for existing or new chemical vapours can be added to the database in order to create new training sets and update the ability of the machine learning model beyond the original training set(s).

FIGS. 11 to 13 describe an exemplary machine learning process.

In a training step of the machine learning process shown in FIG. 11, raw sensor output data is converted to a data set using feature calculation. The raw sensor output data in this case is measured electrical resistance data obtained by the gas sensing apparatus 30. The data set is split into a training set and a validation set, e.g. a 70:30 or 80:20 split. The training set is used to train the machine learning model so that it can determine the features that are predictive for the activity in question. The validation set is then fed to the trained model before being compared with the output predicted values of the trained model for quality assurance purposes.

In an application step of the machine learning process shown in FIG. 12, raw sensor output data is converted to a data set using feature calculation. The data set is then fed into the trained model from FIG. 11 to obtain output predicted values that can be used to determine the sample make-up.

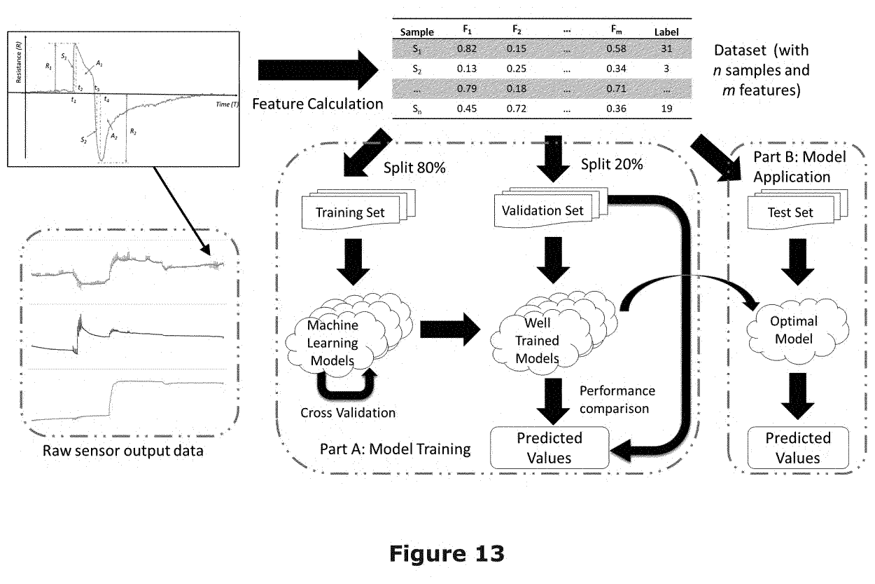

FIG. 13 illustrates the training and application steps of FIGS. 11 and 12 as a single overall machine learning process.

An experiment was carried out in which 34 molecules were selected and tested with the gas sensing apparatus 30. Three different batches of data (I, II, III) were tested, with four samples for each molecule. The batches I and III were merged and used as the training set. The batch II was used independently as the test set. After removing noise and blank samples, 238 and 110 samples were left in the training and test sets respectively.

FIG. 14 shows measured electrical resistance data obtained by the gas sensing apparatus. For each sample, ten features were extracted from the measured electrical resistance data. These features are the maximum electrical resistance (R₁), the largest slope (S₁), the time point of largest slope (t₁), the time point of maximum electrical resistance (t₂), the area of response process (A₁), the minimum electrical resistance (R₂), the smallest slope (S₂), the time point of smallest slope (t₃), the time point of minimum electrical resistance (t₄) and the area of recovery process (A₂), as illustrated in FIG. 14.

Subsequently, the machine learning model constructed for multi-label classification used the ten features as inputs and provided categories of molecules as outputs. Four algorithms were benchmarked for model construction, Random Forest (RF), Support Vector Machine (SVM), Naïve Bayesian (NB), and k-nearest neighbours algorithm (KNN). The RF, SVM, NB and KNN models were implemented through Scikit-Learn. In RF, the number of trees was set as 1000 and the split criterion was set as “gini”. In KNN, the number of neighbor was set as 3. In SVM, a radial basis function (RBF) kernel was used and the parameter space of C and y were set as [2-5, 215] and [2-15, 25], respectively. FIG. 15 shows that the Random Forest algorithm achieved the best performance with very high accuracy levels for both the cross validation and independent test sets.

The inventors found that the use of the machine learning model was successful in providing the gas sensing apparatus 30 with the ability to recognise specific patterns and identify a large number of chemical vapours with high fidelity (whether it be a pure chemical compound or a mixture of chemicals in the gas phase) based on machine-learning pattern recognition. Also, when unknown vapour samples were introduced into the gas sensing apparatus 30, the machine learning model was successful in predicting the chemical nature of the vapour samples.

The trained machine learning model may be used to identify vapours based on previously known chemical fingerprints, independent of the sensor batch. This is made possible by the high reproducibility of the responses of the individual sensors 32 due to the gas sensing apparatus design and the fabrication protocol.

The selectivity of the gas sensing apparatus 30 and its ability to recognise different vapours is therefore made possible through the combination of the different responses of its multiple sensors 32 to the same vapour together with machine-learned recognition of the patterns of the responses.

It is envisaged that, in addition to or in place of electrical resistance, other electrical parameters may be measured. Non-limiting examples of such electrical parameters are described throughout the specification.

Optionally the gas sensing apparatus may be configured so that the measurement device includes a detector configured to measure a change in mechanical parameter (e.g. vibration frequency when the sensors are subjected to excitation) and/or a change in structural parameter (e.g. shape, size) of each polymer layer responsive to interaction of an analyte with the respective polymer layer. Such a detector may be a spectrometer, such as an infra-red spectrometer or a Raman spectrometer. In such embodiments, the graphene sensing layers may be omitted.

Measuring mechanical and/or structural changes in the polymer layers provide an additional or alternative way of using the multiple sensors to obtain a combined set of information that provides a unique chemical fingerprint that corresponds to the presence of the or each analyte in the gas or gas mixture.

It will be appreciated that the fabrication protocol and the above numerical values are merely intended to help illustrate the working of the invention and may vary depending on the requirements of the gas sensing apparatus 30 and the gas sensing application.

The listing or discussion of apparently prior-published document or information in this specification should not necessarily be taken as an acknowledgement that the document or information is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention.

1. A gas sensing apparatus for sensing one or more analytes in a gas or gas mixture, the gas sensing apparatus comprising:
a plurality of sensors, each sensor including a polymer layer, each polymer layer made of a respective different type of chemically non-selective or semi-selective polymer; and
a measurement device configured to measure a change in parameter of each sensor responsive to interaction of an analyte with the respective polymer layer.

2. A gas sensing apparatus according to claim 1 wherein each sensor includes a sensing layer coated with the corresponding polymer layer, and the measurement device is configured to receive an electrical signal from each sensor, the measurement device configured to measure a change in electrical parameter of each sensor responsive to interaction of an analyte with the respective polymer layer.

3. A gas sensing apparatus according to claim 2 wherein the sensing layer is a graphene-based sensing layer.

4. A gas sensing apparatus according to claim 3 wherein each sensor includes a graphene field-effect transistor, and the graphene-based sensing layer is built into the graphene field-effect transistor.

5. A gas sensing apparatus according to claim 1 wherein the measurement device includes a detector configured to measure a change in mechanical parameter of each polymer layer responsive to interaction of an analyte with the respective polymer layer.

6. A gas sensing apparatus according to claim 1 wherein the measurement device includes a detector configured to measure a change in structural parameter of each polymer layer responsive to interaction of an analyte with the respective polymer layer.

7. A gas sensing apparatus according to claim 5 wherein the detector includes a spectrometer.

8. A gas sensing apparatus according to claim 1 wherein each polymer is selected from a group consisting of: polymethyl methacrylate; cellulose acetate butyrate; tetrafluoroethylene-perfluoro-3,6-dioxa-4-methyl-7-octenesulfonic acid copolymer.

9. A gas sensing apparatus according to claim 1 wherein each polymer is a transfer polymer.

10. A gas sensing apparatus according to claim 1 wherein the measurement device includes a processor and memory including computer program code, the memory and computer program code configured to, with the processor, enable the measurement device at least to combine the measured changes in parameter of the sensors so as to generate a chemical fingerprint of the or each analyte in the gas or gas mixture.

11. A gas sensing apparatus according to claim 1 wherein the measurement device includes a processor and memory including computer program code, the memory and computer program code configured to, with the processor, enable the measurement device at least to analyse the measured changes in parameter of the sensors to identify the or each analyte in the gas or gas mixture.

12. A gas sensing apparatus according to claim 11 wherein the memory and computer program code are configured to, with the processor, enable the measurement device at least to analyse the measured changes in parameter of the sensors to identify the or each analyte in the gas or gas mixture by providing the measured changes in parameter of the sensors as input to a machine learning model and identifying the or each analyte in the gas or gas mixture based on an output of the machine learning model.

13. A method of using a gas sensing apparatus to sense one or more analytes in a gas or gas mixture, the method comprising the steps of:
providing a plurality of sensors, each sensor including a polymer layer, each polymer layer made of a respective different type of chemically non-selective or semi-selective polymer;
exposing the plurality of sensors to the gas or gas mixture; and
measuring a change in parameter of each sensor responsive to interaction of an analyte with the respective polymer layer.

14. A method according to claim 13 wherein each sensor includes a sensing layer coated with the corresponding polymer layer, the method including the step of measuring a change in electrical parameter of each polymer layer responsive to interaction of an analyte with the respective polymer layer

15. A method according to claim 14 wherein the sensing layer is a graphene-based sensing layer.

16. A method according to claim 15 wherein each sensor includes a graphene field-effect transistor, and the graphene-based sensing layer is built into the graphene field-effect transistor.

17. (canceled)

18. (canceled)

19. A method according to claim 13 wherein each polymer is a transfer polymer, and wherein the step of providing the plurality of sensors includes using each transfer polymer to transfer the respective sensing layer from a first substrate or surface to a second substrate or surface, wherein the second substrate or surface forms part of the gas sensing apparatus.

20. A method according to claim 13 wherein the step of providing the plurality of sensors includes taking each sensor from a respective different batch of sensors, wherein the polymer layer of each sensor in the respective different batch of sensors is made of a same type of chemically non-selective or semi-selective polymer.

21. (canceled)

22. (canceled)

23. (canceled)

24. A computer-implemented method of identifying one or more analytes in a gas or gas mixture, the method comprising:
collecting a set of data by carrying out the method according to claim 13, wherein the collected set of data includes the measured changes in parameter of the sensors;
creating a training set including the collected set of data;
training a machine learning model using the training set;
identifying the or each analyte in the gas or gas mixture based on an output of the machine learning model.

25. A computer program comprising computer code configured to perform the method of claim 24.
